(19) United States
(12) Patent Application Publication
(10) Pub. No.: US 2011/0040205 A1
(43) Pub. Date: Feb. 17, 2011

(54) Treatment for Alleviating Tinnitus and Hyperacusis with Auditory Stimulation by Compensating for Hearing Loss and Loss of Non-Linear Compressions

(75) Inventors: Lucas C. Parra, New York, NY (US); Barak A. Pearlmutter, Dublin (IE)

Correspondence Address: COHEN, PONTANI, LIEBERMAN & PAVANE LLP, 551 FIFTH AVENUE, SUITE 1210, NEW YORK, NY, 10176, US

(73) Assignees: The City University, New York, NY (US); University of Ireland, Maynooth, County Kildare (IE)

(21) Appl. No.: 12/741,743

(22) PCT Filed: Nov. 10, 2008
(86) PCT No.: PCT/US08/12687

§ 371 Date: Aug. 26, 2010

Related U.S. Application Data
(60) Provisional application No. 61/002,786, filed on Nov. 9, 2007.

(51) Int. Cl. A61B 5/12

(52) U.S. Cl. 600/559

(57) ABSTRACT

An auditory stimulation treatment for alleviating tinnitus and hyperacusis by compensating for hearing loss and loss of non-linear compression. Natural auditory signals are delivered correcting for hearing loss and compressive non-linearity, both determined at separate frequency bands for the individual subject. This differs from existing treatment in that synthetic “masking” signals are not delivered, but rather the natural auditory input to the subject is modified. Consequently, the method is more akin to a conventional hearing aid. In contrast to conventional hearing aids, however, perception thresholds are specifically corrected and non-linear compression is matched to the specific hearing deficit of the user.

### CROSS REFERENCE TO RELATED APPLICATIONS

The present application claims priority from U.S. Provisional Patent Application Ser. No. 61/002,786 filed on Nov. 9, 2007, the contents of which is incorporated in its entirety.

### BACKGROUND OF THE INVENTION

1. Field of the Invention

The present invention relates to a treatment for alleviating tinnitus and hyperacusis and, more particularly, to a method for alleviating tinnitus and hyperacusis with auditory stimulation by compensating for hearing loss and loss of non-linear compression.

2. Description of the Prior Art

Tinnitus is the perception of a phantom sound often associated with hearing loss. Mild tinnitus is rather common, reported by many subjects after a few minutes in a quiet environment. The subjective sound varies, often described as a “buzz”, “ring”, “hiss”, “hum,” or the like. Severe tinnitus is almost always indicative of hearing loss, with the pitch of the phantom sound generally corresponding to the frequencies of hearing loss.

To date, a variety of therapeutic approaches to alleviate tinnitus have given mixed results. It is therefore generally assumed that tinnitus may be the result of multiple physiological causes. It is believed that in most cases, the tinnitus percept does not immediately originate at the cochlea. Instead, it has often been associated with adaptive phenomena in the central nervous system. A variety of models for the physiological origin of this form of central tinnitus have been proposed.

The Zwicker tone is an auditory perceptual illusion named after the scientist who first characterized the tone. The Zwicker tone is a transient phantom or illusory sound that is perceived by most subjects after perceiving a notched broadband signal. The frequency of the illusory sound is within the notched frequency band. The strength and duration of the Zwicker tone percept depends on stimulus conditions and is quite variable across subjects. Despite their apparent similarity, the relationship between the Zwicker tone and tinnitus is not well established.

Gain and contrast adaptation is a common strategy of the perceptual system to match a large dynamic range of natural signals to the limited dynamic range of sensors and neurons. Perhaps the best known gain adaptation mechanism is the closing of the iris of the eye when stepping from a dark room into bright sunlight. The analogous effect in hearing is the acoustic middle-ear reflex that mechanically attenuates sound transmission to the cochlea in response to loud sounds.

Adaptation to changes in stimulus statistics is a ubiquitous and long studied phenomenon in the nervous system. Visual neurons in the retina and visual cortex adjust the gain of their transfer functions to maintain a high sensitivity at varying luminance contrast levels. This allows the visual system to operate well under drastically varying external conditions. In the auditory system, adaptation is observed at various levels. Efferent feedback to outer hair cells are thought to control the gain of cochlear amplification, while auditory nerve fibers are known to adapt their firing rate at various time scales. Finally, inferior colliculus neurons have been shown to adjust their response thresholds and gains to optimally encode variations in the auditory stimulus.

The cochlea transforms acoustic signals into neuronal activity by decomposing the signal into its various frequency components that are then transmitted by the auditory nerve to the midbrain. The signal intensity in different frequency bands is encoded in the firing of different auditory nerve neurons. The dynamic range of the external stimuli, however, is known to be much larger than the dynamic range of this neuronal activity. To transmit auditory information through this information bottleneck, adaptive mechanisms are therefore required. The nervous system has developed various strategies to cope with this problem, including in particular, gain adaptation.

There are numerous known techniques for treating tinnitus with auditory stimulation. For example, one investigator, Arnaud Norena, performs compensatory stimulation using synthesized sounds. Specifically, Norena performed high frequency auditory stimulation in an attempt to compensate for high frequency hearing loss, which is the most common type of hearing loss. Norena's work is limited to adjusting the delivered sound based on loss in hearing sensitivity as assessed by perception thresholds measurements. However, hearing loss as assessed with perception thresholds is an insufficient predictor of tinnitus, and hence compensating loss of sensitivity alone may not be sufficient to compensate the peripherial hearing deficit associated with tinnitus. In contrast to the method proposed by Micheyl and Norena in U.S. Pat. No. 6,974,410 the present invention attempts to measure and compensate for loss of non-linear compression often found in tinnitus subjects.

A similar approach to treat tinnitus with auditory stimulation is currently being commercialized by Neuromonics. Their approach is similar in that sounds delivered to the subjects are adapted in response the measured audiogram (hearing thresholds at different frequencies). Efficacy of their treatment paradigm has recently been published. (Henry J A, Zaugg T L, Myers P J, Schechter M A. Using therapeutic sound with progressive audiologic tinnitus management. Trends Amplif. 2008 September; 12(3):188-209. Epub 2008 July 29; Hanley P J, Davis P B. Treatment of tinnitus with a customized, dynamic acoustic neural stimulus: underlying principles and clinical efficacy. Trends Amplif. 2008 September; 12(3):210-22; Davis P B, Wilde R A, Steed L G, Hanley P J. Treatment of tinnitus with a customized acoustic neural stimulus: a controlled clinical study. Ear Nose Throat J. 2008 June; 87(6):330-9. Davis P B, Paki B, Hanley P J. Neuromonics Tinnitus Treatment: third clinical trial. Ear Hear. 2007 April; 28(2):242-59).

Even though these techniques may be suitable for specific individuals they do not fully exploit the potential of auditory stimulation to alleviate tinnitus and hyperacusis. In particular, they do not address the deficit in non-linear compression found in many tinnitus subjects. The disclosed method aims to address this need in an effort to improve efficacy of auditory stimulation expand its applicability to a larger tinnitus population.

### SUMMARY OF THE INVENTION

Disclosed is a method for alleviating tinnitus and hyperacusis with auditory stimulation that compensates for hearing loss and loss of non-linear compression and thus improves on the limitations of the prior art.

Natural auditory signals are delivered that correct hearing loss and compressive non-linearity, which are both determined at separate frequency bands for the individual subject. The disclosed method differs from conventional treatments in that synthetic “masking” signals are not delivered but, rather, the natural auditory input to the subject is modified. In this regard, the method is more akin to a conventional hearing aid. In contrast to conventional methods associated with hearing aids, however, perception thresholds are specifically corrected and non-linear compression is matched to the specific hearing deficit of the user.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are merely intended to conceptually illustrate the structures and procedures described herein.

### BRIEF DESCRIPTION OF THE DRAWING

The foregoing and other advantages and features of the invention will become more apparent from the detailed description of the preferred embodiments of the invention given below with reference to the accompanying drawings in which:

FIG. 1 is a graphical plot of the outputs of each processing step of the tinnitus-like percept being generated by gain adaptation;

FIG. 2 illustrates tinnitus and Zwicker tone in the reconstructed signal and at earlier states of processing according to the model;

FIG. 3 illustrates the effect of perceptual frequency scale on the various stages of auditory processing in the model and on the reconstructed signal;

FIG. 4 illustrates the dependence of reconstruction on noise magnitude, signal loss, and power-averaging time constant; and

FIG. 5 are spectrograms illustrating a test signal used in the listening experiment (second row, with greater detail in the first row) along with the model prediction (third row).

### DETAILED DESCRIPTION OF THE EXEMPLARY EMBODIMENTS

### A. Background on Tinnitus as a Result of Gain Adaptation

Phenomena resembling tinnitus and Zwicker phantom tone can occur when an auditory gain adaptation mechanism attempts to make complete use of a fixed-capacity channel. In the case of tinnitus, the gain adaptation enhances internal noise of a frequency band that would otherwise be silent due to damage. This would generate a percept of a phantom sound as a consequence of hearing loss. In the case of Zwicker tone, a frequency band is temporarily silent during the presentation of a notched broad-band sound which causes a percept of a tone at the notched frequency when the stimulus is terminated.

In a model, the same mechanism leads to a transient phantom percept following the notched sound. The model predicts that the Zwicker tone should be abolished by a short masking sound. The present inventors verified this prediction by performing a psychoacoustic study using short masking sounds following notched noise. The subjective responses match the prediction for subjects with self-reported tinnitus, but do not predict the responses of subjects with normal hearing. Tinnitus often coincides with loss of non-linear cochlear amplification. When logarithmic amplitude compression was included during the verification, which is typical for normal hearing subjects, the model predicted minimal masking of the Zwicker percept by a brief tone. Thus, the model explains the different results for normal and tinnitus subjects by a loss of instantaneous non-linear compression.

The psychoacoustic experiment permitted the establishment of a first empirical link between the Zwicker tone percept and tinnitus. Together with the modeling results, it supports the theory that the phantom percept is a consequence of a central adaptation mechanism confronted with degraded sensory apparatus performing below the level for which it was designed. The hypothesis makes predictions on the relation of distortion products (a byproduct of non-linear cochlear amplification) and Zwicker tone masking, which can easily be tested experimentally. Finally, nonlinear compression is easily measured and can be restored using compressive hearing aids. The present inventors have determined that the foregoing provides a basis for a straightforward diagnosis and treatment option for those cases of tinnitus that can be linked to this particular form of hearing deficit.

The present inventors have developed a conceptual model that has been mathematically validated, which provides a uniform explanation for both phenomena. The conceptual model robustly predicts a link between tinnitus and Zwicker tone, and psychophysical data matches the prediction of the model. The psychophysical data support the proposed model, but also considered in isolation, itself constitutes a novel empirical link between tinnitus and the Zwicker tone. The standard theoretical understanding of simple types of adaptation mechanisms matches sensory statistics. The model of auditory adaptation is based on these principles and provides a mathematically representation of the model. The mathematical model predicts percepts under a variety of conditions. These predictions allow testing of the model experimentally.

After gain normalization, the response in separate frequency bands does not distinguish long term silence from persistent uniform noise. For an efficient information transmission, it is better to transmit overall loudness as a separate variable that can then be used to differentiate silence from uniform noise. The question that begs answering is “what is the neuronal substrate for such a representation?” There are many cells in the auditory cortex with high spontaneous activity responding only transiently with an increase in firing rate to the onset of sound. Only few cortical cells respond tonically to a steady stimulus. A distinct representation between loudness and modulation may therefore not seem unreasonable for the auditory cortex. The situation for the auditory nerve is less straightforward. On the surface it would seem that loudness is encoded in the overall firing rate. Yet, in fact, an increase in firing rate does not necessarily reflect a growth in loudness, and firing rate is not a sufficient model to explain level discrimination. Instead, other mechanisms, such as synchrony and phase relations across fibers, may be required to explain psychophysical performance. It is also conceivable that outer hair cell afferent fibers, which are only recently being characterized, subside an important role in this regard. Despite considerable ongoing efforts, the details of how overall level is encoded in the auditory nerve remains an open question.

The method of the invention operates to process separately each frequency band. No implementation of lateral suppression or any other mechanisms across bands are likely to operate at various levels of auditory processing. As a result, some aspects of the tinnitus and Zwicker tone percept are not captured by the model. For instance, the available reports on the Zwicker tone have suggested that the phenomena is asymmetric. Subjects tend to match the perception with a tone that is closer to the lower edge of the notched band. In fact, a high pass band edge may not elicit a Zwicker tone. Similarly, tinnitus seems to be strongest for hearing loss with sharp bands edges. These phenomena may be explained by lateral inhibition, in particular asymmetric inhibition whereby higher frequency tones inhibit the response of lower frequency channels. Such asymmetric inhibition has been observed at the level of the auditory nerve and cochlear nucleus. The method of the invention is therefore not intended to reproduce these features in detail and avoids introducing additional processing stages as in the Zwicker tone model of Franosch et al. The inventors believe that these effects are not required to explain, and may in fact obscure, the basic functionality of the disclosed method. Rather, the goal is to give a simple explanation for a basic way to derive experimental predictions could.

Taken together, the experimental and modeling results support the theory that tinnitus is a consequence of a gain adaptation mechanism that is confronted with hearing loss and an associated loss of non-linear compression. Specifically, a generic argument that may be applicable as it is predicted that there is a relationship between the strength of non-linear auditory phenomena, such as combination tones, and the masking behavior of the Zwicker tone.

The main theoretical contribution is to demonstrate that some illusory auditory percepts can be explained as direct consequences of gain adaptation and internal noise in the presence of hearing loss. Gain adaptation and noise are basic features of the auditory processing stream. Since gain adaptation may operate at various levels of processing, a simple and generic model is constructed. It is shown that after gain adaptation, frequency bands with reduced external input (due to permanent hearing loss or temporary deprivation) show enhanced steady-state activity resembling phantom sounds.

In accordance with the method of the invention, a generic argument is provided that may be applicable at many stages of auditory processing and, thus, there is a refrain from suggesting which area or areas actually subserve this functionality. No discussion of the auditory nerve as one potential site where this mechanism may play a role is presented. The model associated with the method of the invention is sufficiently generic that it is expected that similar phenomena is to be exhibited by any system in its broad general class. Here, systems perform local gain adaptation in the context of a global estimate of the stimulus energy.

The main goal of the adaptive processing is to transform the signal in different frequency bands into independent channels with optimally matched dynamic ranges. Here, gain adaptation accomplishes two tasks. First, gain adaptation adjusts signal variance to the effective dynamic range of each frequency channel, thus optimizing the information capacity in each frequency channel. Second, gain adaptation removes redundancy across channels. Most acoustic signals have significant redundancy across frequency bands due to the simultaneous increase and decrease of amplitude in multiple bands. In fact, humans can understand spoken language with as few as four distinct frequency bands. By normalizing signal power, channels become more independent. A similar mechanism for reducing redundancy by divisive normalization has been proposed for visual processing and can be used for image compression.

A channel with a fixed dynamic range will communicate maximum information if the transfer function matches the cumulative density function (CDF) of the input variable. In particular, the threshold and slope of the transfer function should match the mean and variance of the data. By adjusting the mean and/or variance of the input, it becomes possible to optimize the transmission for a given transfer function. The first order correction is achieved by adjusting the variance of the signal to match the transfer function.

The simplest estimate of signal power is a running average. Using a discrete-time formulation for simplicity, at time t (in a.u.) at frequency band b (in perceptual units) as defined by

$$¿ ( b , t ) = {\sum }_{i = 0}^{∞} {τ ( 1 - τ )}^{i} / X • b , t - i Δ t {•}^{2} \qquad ( Eq . 1 )$$

where Ät is the time constant of integration. This can be implemented efficiently by a simple update,

\hat{P}(b,t)=(1−τ)\hat{P}□b,t−Δt−Δt□+τ|X(b,t)|² (Eq. 2)

The equalization gain for each band is defined as

G(b,t)= (Eq. 3)

The equalized power can then be defined in accordance with the relationship

|E(b,t)|²=G(b,t)|X(b,t)|² (Eq. 4)

The signal power is defined by |X(b,t)|². Here, an assumption is made that this power is transduced by the cochlea into a neuronal signal, and that transduction and/or neuronal transmission have some inherent noise, albeit perhaps small. For simplicity, it is assumed uncorrelated noise, in which case the noise power |N(b,t)|², can be added to the perceived signal powers |S(b,t)|²

X(b,t)|²=|S(b,t)|²+|N(b,t)|² (Eq. 5)

These simple assumptions give rise to illusory percepts resembling tinnitus and Zwicker tone in response to a reduced input in a given frequency band.

### B. Sensitivity, Hearing Loss, and Perceptual Frequency Scale

The perceived signal intensity in each frequency band is affected by the sensitivity of the cochlea at that band. This is expressed by some gain function h (b), and use h (b) S (b,t) instead of S (b, t). Hearing loss is modeled by reducing h (b) for the damaged bands. It is noted that this simple model is linear in power and does therefore not include the non-linear compression typically found for an intact cochlea. The model therefore resembles the sharper increase in firing rate with increasing signal power observed for the damaged cochlea. The broadening of the bandwidth associated with hearing loss, however, has not been modeled.

The presented inventors also tested the relevance of basic psychoactoustic properties, such as perceptual frequency resolution. Filter bank gains w (b, f) relating the acoustic signal intensity S (f, 1) at frequency f to the perceived signal intensity S (b, f) at perceptual frequency band b are introduced. Together with the sensitivity h (b) the following relationship holds true:

$$S ( b , t ) = h ( b ) {\sum }_{f} ω ( b , f ) S ( f , t ) \qquad ( Eq . 6 )$$

Results are presented on a simple linear frequency scale, b=f, as well as the perceptual Equivalent Rectangular Bands (ERB). In this model, the non-linear frequency scale alters the spectral profile of tinnitus, but are not required for its generation.

### C. Recovered Signal

To interpret the neuronal representations after gain adaptation, they are used to construct an estimate of the original signal. This step may seem artificial as the nervous system does not need to regenerate the original signal to perceive it. Rather, the neuronal representation itself is the equivalent or precursor of perception. If the representation is altered so that the stimulus cannot be regenerated, even approximately, then the percept must be equivalently distorted, and that the reconstruction technique provides an intuitive way to measure and visualize the distortion of the neuronal representation.

This method allows seeing that the regenerated signals after gain adaptation exhibit artifacts that would be perceived as phantom sounds.

To interpret (hear) the adjusted signal, reproduction of the original signal S (f, t) from E (b, t) is attempted. The inverse of the linear transformation U (b,f) is applied, which is denote by v (f, b), and use the total power of the signal P (t) is used.

S(f,t)=P(t)Óv(f,b)E(b,t) (Eq. 7)

The assumption here is that the system is provided with no knowledge of the varying gain it has applied to the signal. The system does know, however, how to interpret its permanent filter bank responses, and it does know the overall loudness of the signal P (t).

Gain normalization as proposed removes the common power of the signal on the time scale τ, i.e. the overall loudness of a signal is therefore no longer reflected in the individual perceptual channels. Silence lasting longer time scales would therefore be indistinguishable from loud uniform noise. Consequently, the common signal power P(t) must be separately encoded. For a frequency co-modulated signal, power is redundantly distributed across bands. Removing this co-modulation removes the redundancy and makes a mere efficient use of the information capacity of the channel. Communicating overall power, as a variable separate from the power fluctuation in each frequency band is therefore a more efficient use of channel information capacity.

Also noted is that the linear transformation a may not be invertible. In fact, the large bandwidths at high frequencies in the perceptually realistic Equivalent Rectangular Bands (ERB) scale precludes inversion. In that case, a regularized pseudo inverse is used. Here, recover S (f, t) even in the case of zero noise and no hearing loss can thus only be approximated.

Finally, to be able to listen to the recovered signal, the time domain signals from its frequency powers must be regenerated. The powers give amplitude, but not phase information. This is a common problem in speech and sound synthesis. A standard engineering solution to this problem is to reuse the phase that was obtained when analyzing the original signal. If the powers have not changed significantly, the resulting signal is perceptually similar to the original.

### D. Modeling Results

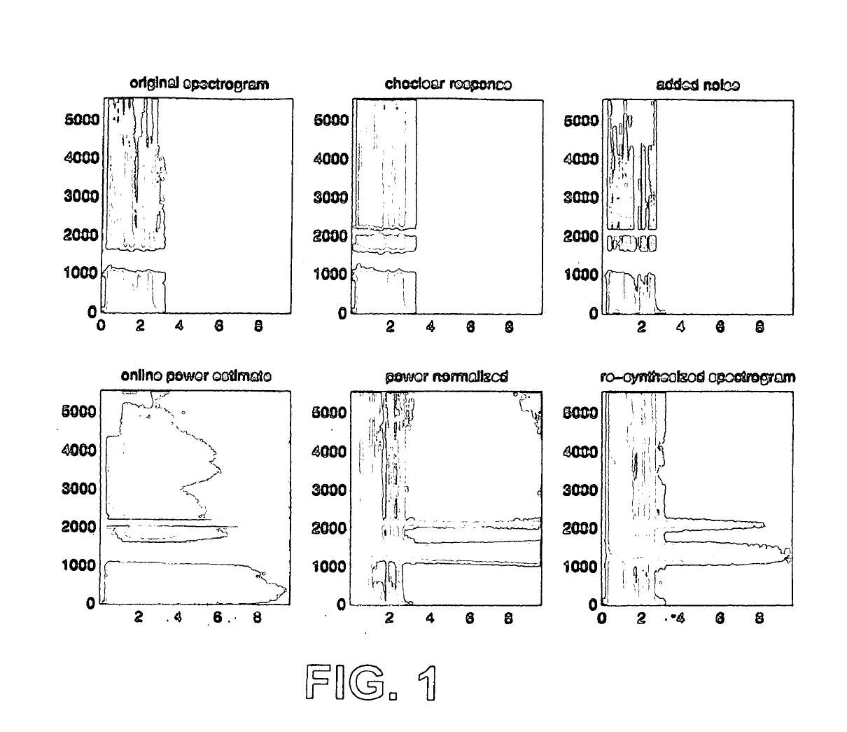

In accordance with the method of the invention, the present inventor simulated hearing damage by reducing the sensitivity h (t) in a narrow frequency band. FIG. 1, which illustrates the outputs of each processing step of the tinnitus-like percept being generated by gain adaptation, shows the result for a 60 dB hearing loss at 3 kHz and −30 dB internal noise. This simulation uses a linear frequency scale with linearly increasing sensitivity (to match a typical 1/f power spectrum). The bottom right panel shows that gain adaptation generates steady state power at the damaged frequency band. The reconstructed signal contains a sound similar to tinnitus.

With further reference to FIG. 1, the auditory signal is first decomposed into a time frequency representation. Frames of 16 ms (256 samples at 16 kHz sampling rate) around time t are windowed with a Hanning window and Fourier transformed to obtain 128 frequency amplitudes |S(f,t)| (shown top left) and phase arg(S(f, t)) (not shown). Image intensity in this, and other figures, represents power in dB. Time t in seconds varies on the horizontal axis. Frequency varies on the vertical axis up to the Nyquist frequency. Perceptual amplitudes |S(b,f)| (shown top center) are computed with Equation (6). Noise with a 1/f power profile (|N(f,t)|²á 1/f) is added to the perceived powers giving the signal (|X(b,t)|² 12 (shown top right) following Equation (5). The gain and equalized signal powers (shown bottom left and center) are computed with Equations (3) and (4) using a time constant of τ=1 sec. Finally, the original signal powers are estimated from this activity using Equation (7). To synthesize the signal, a conventional overlap-add procedure is used. Powers are combined with the original phase arg(S (f, t)), inverse Fourier transformed, multiplied with a Hanning window, and added in half overlapping frames. A spectrogram of this re-synthesized signal is shown on the bottom left.

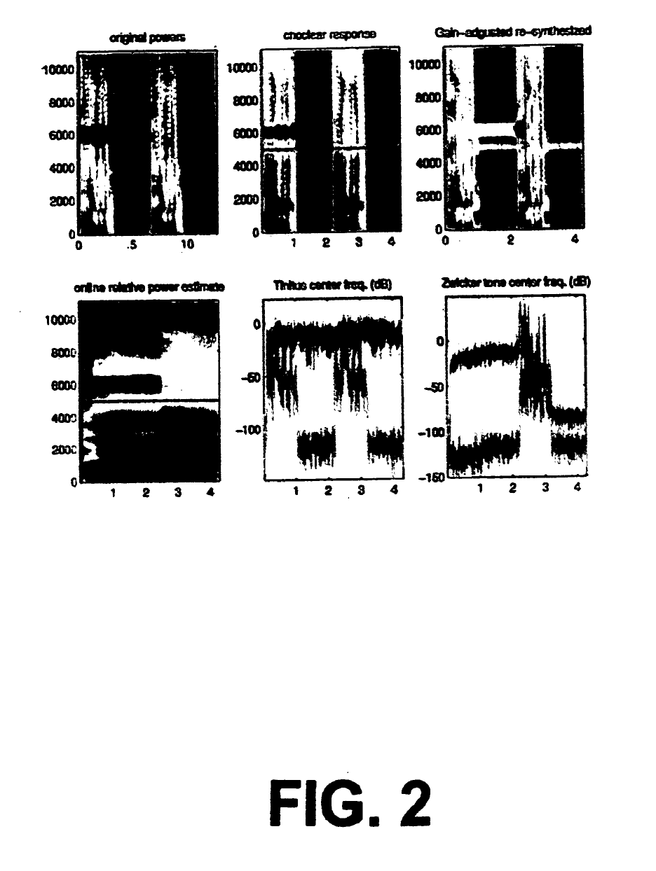

FIG. 2, which illustrates tinnitus and Zwicker tone in the reconstructed signal and at earlier states of processing in accordance with the method of the invention, shows the results obtained for a broadband sound with a notched response (power reduced by 60 dB at kHz). Power normalization fills in the gap and generates an artificial tone following the notched noise. This is consistent with the Zwicker tone phenomenon. The phantom sound is immediately aborted upon presentation of an auditory signal in that frequency band. It is predicted that the Zwicker tone can be similarly aborted by a brief signal in the corresponding frequency band.

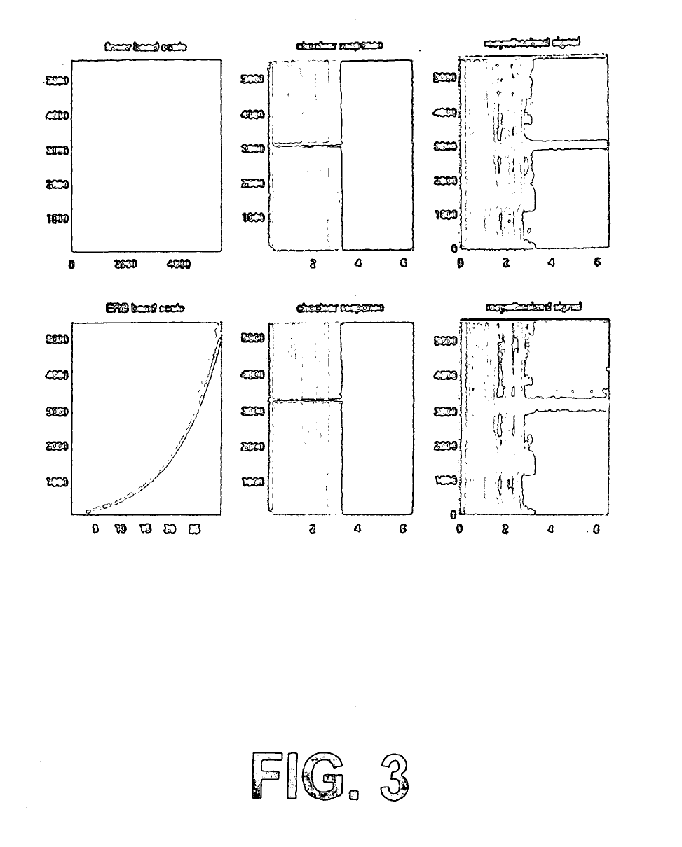

The effect of the perceptual sensitivity profiles on the generation of tinnitus in the method were compared. FIG. 3, which illustrates the effect of perceptual frequency scale on the various stages of auditory processing in the model and on the reconstructed signal, shows the results obtained with a linear frequency band and an ERB scale. The re-synthesized signal for the ERB scale shows a broader phantom sound with a number of side bands. The broadening is a result of the broad bands on the perceptual scale. It is speculated that the difficulty of human subjects in matching synthetic tones to their percept of tinnitus, may be due to this more complex structure resulting from a damaged band.

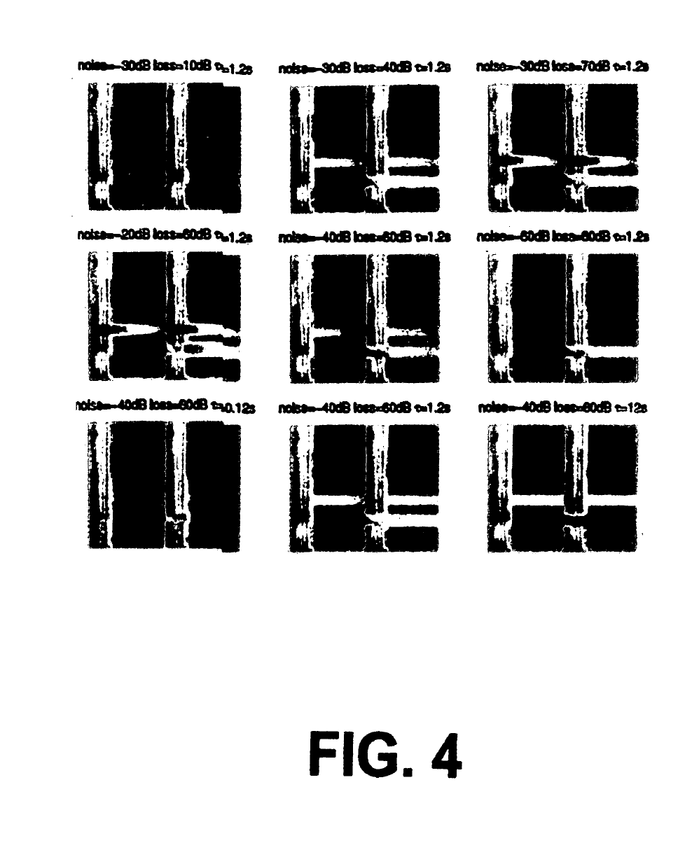

In the simple case of the linear frequency scale, the model has only three, free parameters: (i) the time integration constant τ⁻¹; (ii) the level of hearing loss; and (iii) the amount of internal noise. FIG. 4, which illustrates the dependence of reconstruction on noise magnitude, signal loss, and power-averaging time constant, shows the effect of each of these parameters on the phantom sound. The intensity of the phantom sounds increases with the level of internal noise and with the loss of signal intensity. The intensity is fairly independent of r.

### E. Model Predictions and Evaluations

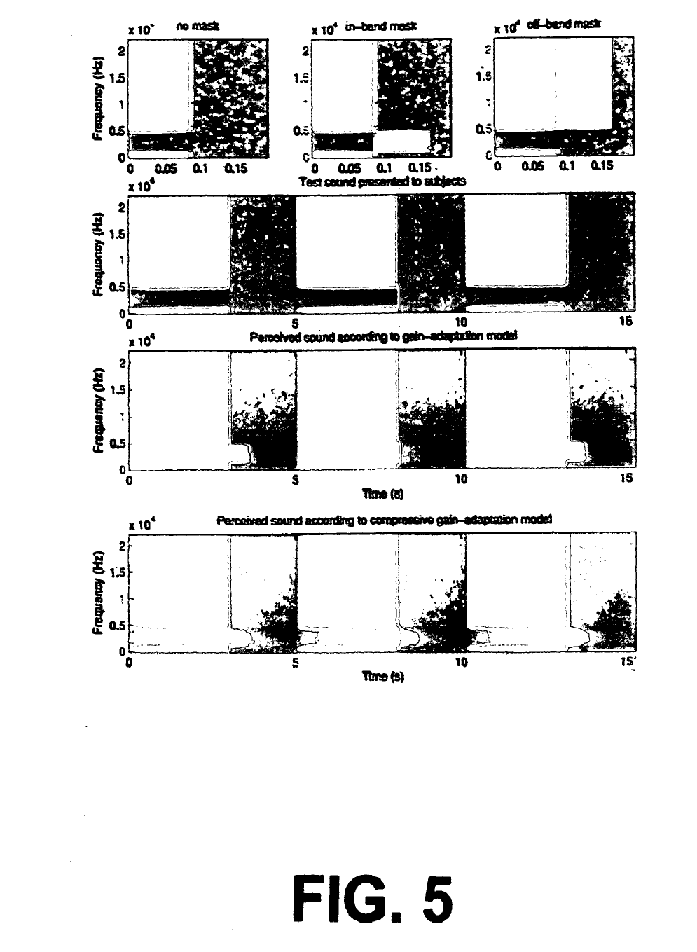

The modeling results shown in FIG. 2 (top, right panel) indicate that the Zwicker tone percepts is attenuated as soon as a signal in the corresponding frequency band is presented. It is predicted therefore that the Zwicker phantom tone percept can be aborted by a brief “masking” sound in the notched frequency band. To test this prediction, a listening experiment in which subjects had to evaluate the presence and/or strength of a phantom tone in response to a notched noise followed by a masking sound was created. The masking sound was a short broad-band noise covering either the notched band (in-band) or a band above or below the notched bang (off-band) as shown in FIG. 5, which are spectrograms illustrating a test signal used in the listening experiment (second row, with greater detail in the first row) along with the model prediction (third row).

With further reference to FIG. 5, the gain adaptation model predicts that an in-band masker (following the second notched noise in this example) will abort the Zwicker percept, whereas an off-band masker (following the third notched noise) will not alter the phantom percept. When a compressive non-linearity is included in the model (bottom row), the Zwicker tone is weaker and only weakly attenuated by the short in-band mask.

The experiment was created as a two-alternative forced choice task. Here, subjects were presented by a pair of notched noise sounds, each followed in random order either by an in-band mask or an off-band mask. Subjects had to decide which of the two sounds elicited a stronger phantom percept. The model predicts that subjects would answer in favor of the noise followed by the off-band mask in every case since only an in-band mask would reduce the elevated gain leading to the phantom percept.

The experiment requires that participants perceive the Zwicker phantom tone. Since the percept is quite variable across subjects, the present inventors were required to first determined whether a given subject perceived the phantom sound. A group of subjects, such as twenty (20), reported different percepts describing them as a “tone”, “hiss”, or “ringing” lasting a brief moment after the notched noise. The majority of subjects (e.g., 14 out of 20) perceived a sound of varying strength for different notched-bands, while a few did not perceive a phantom tone following any of the notched noise sounds (e.g., 6 out of 20). None of the subjects perceived a phantom sound in the control condition of white noise with a flat spectrum.

All subjects were asked if they perceive in their daily lives spurious ringing on a regular basis, to which an exemplary 6 subjects responded positively. There was a correlation between this self reported tinnitus and the ability to perceive the Zwicker tone with the stimuli (τ=0.48, p=0.03). None of the exemplary 6 subjects failing to hear a Zwicker tone reported habitual tinnitus. The tinnitus percept, however, was not a prerequisite for perceiving the Zwicker tone. Of the exemplary 14 subjects that perceived the Zwicker tone, only an exemplary 6 reported tinnitus.

The Zwicker tone masking experiments with these 14 exemplary subjects was performed. A significant correlation between the subject responses and the model predictions for 9 of these 14 exemplary subjects with correlation coefficients ranging between 0.6 and 0.9 and corresponding p-values in the range of 10⁻² to 10⁻⁷ was measured. It was found that the model's prediction coincided with the responses of all subjects that did report tinnitus, and only failed for subjects that did not report tinnitus. In essence, the perception of tinnitus was a perfect or ideal predictor for the subjective response to the notched noise in isolation and when followed by a short masking sound (6 out of 6).

The foregoing results indicate that the model as described so far is suitable for tinnitus subjects, but is not adequate for normal hearing subjects. An important deficit often associated with hearing loss, and reported sometimes also for tinnitus, is the loss of a compressive non-linear amplification. The present model did therefore not include any non-linear compression. In contrast, normal hearing subjects showed a logarithmic sensitivity in their perception of signal power. Therefore, the model was modified to include a logarithmic compression by using log-powers log (|X(b,t)|² instead of powers (|X(b,t)|² in equations (1)-(4).

FIG. 5 (bottom row) shows that with this modification a method is obtained that produces again a phantom tone following a notched noise. In contrast to the non-compressive model, however, this illusory percept is somewhat weaker and only minimally attenuated by the short masking sound used in the experiment. This is in agreement with the observation that some normal hearing subjects did not perceive the Zwicker tone, while normal-hearing subjects that did perceive the tone, typically heard no difference for the two different masking conditions.

### F. Methods

### Psychophysics

(1) Subjects

The above described 20 volunteers (10 male, 10 female, age 26±7) were recruited among faculty and students at The City College of New York (CCNY) in accordance with the CCNY IRB guidelines. Subjects provided their informed consent prior to experimentation. In the sample, there was no significant correlation between the subject's age and self-reported tinnitus, but there was a weak correlation between age and the Zwicker percept (τ=0.48, p=0.02). The presence of the Zwicker percept was determined with the following procedure.

(2) Zwicker Perception Test

Prior to the Zwicker masking experiment, if subjects reliably perceive a phantom tone by presenting four different noise sounds in random order (the control sound was white noise and the other three were notched noise with different notch bands as described below) were tested. The subjects were instructed to report after which of the four noises they heard some form of ringing, however faint it might have been. The percept was considered as factual if the subject reported consistently a percept for the same notched sounds (despite the random ordering) but not the white noise. Subjects that did not report any phantom precept or who gave inconsistent answers to this test would not be able to perform the perceptual discrimination and were, therefore, excluded from the masking experiment.

(3) Phantom Tone Masking Experiment.

FIG. 5 shows an example of the tones sequence that was presented to the subjects in the main experiment. The first notched sound was presented as a reference signal to help subjects identify the Zwicker phantom tone at a given frequency. The same notched noise is repeated, for example, two times, and is followed each time by an in-band mask or by an off-band mask in random order. The task for the subject is to judge which of the two repetitions of the notched noise was followed by a stronger phantom tone percept. Subjects were instructed to use the first notched noise as a reference for their judgment. Subjects selected their level of confidence on a continuous scale from 1 to 2, choosing 1 if they were confident that the first repetition elicited the stronger phantom percept, 2 for the second, and intermediate values if they were less confident about their choice. The reasons for uncertainty included not perceiving the phantom tone at all or that the phantom tone was perceived for both repetitions with similar strength. A total of 18 noise sound triplets were presented for judgment. Subjects reported that this task was not easily performed, which was reflected in many intermediate ratings. The predictions of the model where labeled as 1 or 2 according to which sound was followed by the in-band mask, and the correlation of the model and subject responses were calculated.

(4) Stimuli

The amplitude of the notched noise raises linearly within 1000 ins, holds for 1000 ins, and decays within 40 ins. The noise mask raises and decays linearly within 40 ins, lasting a total of 80 ins. The band-gap of the notched noise was 4 KHz wide, starting a 500, 1000, or 2000 Hz. The in-band masks utilizes the same parameters as the off-band mask. The off-band masks are either below or above the notched band. For example, the notched band starting at 500 Hz has an in-band mask covering 500 Hz to 4500 Hz, the lower off-band masks covers 0 Hz to 500 Hz, and the higher off-band mask covers 4500 Hz up to 22050 Hz, which is the Nyquist frequency for the signals associated with the experiment. The mask sounds were calibrated in amplitude to be perceived with equal loudness as compared to white noise and delivered at −30 dB relative to the notched noise. The signals were generated on a PC using MATLAB by zeroing the corresponding frequencies in the Fourier domain. They were reproduced using an external USB digital-to-analog converter, such as an Audiotrack MAYA44 USB external digital-to-analog converter, and delivered binaurally with headphones, such ATH-M40f manufactured by audio-technica, at approximately 50-60 dB SPL, adjusted for comfort.

### G. Discussion

Illusory visual percepts were once thought to constitute regimes where the visual system breaks down and fails to process the data appropriately. For a number of broad classes of stimuli, this is no longer the accepted explanation. For example, many motion illusions can be explained as a consequence of Bayesian inferences being made from noisy images. This theory has been extended to the auditory system, where it is proposed that a simple adaptive mechanism, when driven outside its normal operating regime, may generate illusory percepts. Specifically, the psychophysics and modeling results support the hypothesis that tinnitus and the Zwicker tone may be a consequence of gain adaptation, and that the loss of compressive non-linearity may accentuate and modify these percepts, even in the absence of elevated hearing thresholds.

The results of the listening experiment could also be explained with conventional forward masking operating on a time scale of 100-200 ins, i.e., the tone after the notched noise masks the Zwicker percept. In fact, the added tone has been called a masking tone. This interpretation is not necessarily disagreed with, since after all forward masking has traditionally been explained with gain adaptation. Conventional masking, however, does not explain the different results for normal and tinnitus subjects.

Instead, as already seen, the present inventors have established by experiment a distinct regime of operation for tinnitus subjects. It might be speculated that tinnitus subjects have lost the instantaneous amplification mechanism of outer hair cells in selective bands. This disrupts the dynamic range compression inherent in the non-linear amplification mechanism. As a result, a slower neuronal gain adaptation mechanism becomes the dominant factor.

### H. Evidence Related to Tinnitus and Hearing Loss

In the auditory periphery there are at least two mechanisms that are thought to address the problem of dynamic range mismatch between the auditory nerve fibers, which lies between 20-40 dB, and the dynamic range of 120 dB in the auditory input. First, outer hair cells are thought to actively amplify faint sounds with large gains, while for large signal intensities the gain is reduced. This non-linear amplification leads to a compression of dynamic range. Second, inner hair cells are contacted by multiple auditory fibers with different response thresholds and gains. Therefore, as intensity increases an increasing number of fibers are recruited, which effectively increases the available dynamic range of neuronal firing for a group of fibers with common characteristic frequency.

Peripheral hearing loss is associated with elevated thresholds. This results in a reduced diversity of response thresholds required by the recruitment mechanism. This is thought to be the origin of abnormally first growth in loudness. In addition, outer hair cell damage, which is often associated with peripheral hearing loss, leads to a loss of active amplification, reducing the compressive effect on the non-linear cochlear amplifier. In the face of these challenges, it is postulated that downstream mechanisms take a bigger burden in coping with the dynamic range of the input. These mechanisms, when confronted with silence in selected frequency bands increase internal gains, which then amplify neuronal noise to be perceived as phantom sounds. Tinnitus and the Zwicker tone in this view are not reflected by increased activity in the periphery, but may be observed more centrally, yet they are caused by alterations in the peripheral apparatus.

Elevated thresholds are a common correlate of tinnitus, and abnormal growth of loudness is observed for frequencies marching the tinnitus percept. In addition, distortion products, which are thought to reflect the operation of the non-linear cochlear amplifier, are selectively altered for frequency bands having been matched to the tinnitus percept. Finally, release from masking by a secondary masking tone does not occur in tinnitus subjects, indicating once again that the non-linear effect of two tone suppression ascribed to the cochlear amplifier is not operating in tinnitus subjects. All this supports the theory that tinnitus is a result of hearing loss and degraded non-linear compression.

A common strategy to alleviate tinnitus consists in masking the tinnitus percept with acoustic noise in the corresponding frequency band. While this method is effective in eliminating the tinnitus percept for the duration of the noise, it is seldom adopted by patients as it accomplishes little more than replacing one disturbance with another. Interestingly however, a residual inhibition following the masking noise and lasting up to minutes is commonly observed. It has also been reported that hearing aids properly fitted to the frequencies of hearing loss can alleviate tinnitus at those frequencies. Some reports indicate that tinnitus can be alleviated on a longer time scale by delivering variable signals in selected frequency bands. All this is in perfect agreement with the theory that the increased gains can be reduced by delivering signal variance to the damaged channel. It suggests that a properly fitted compressive hearing aid, such as commonly available in the market, may in fact alleviate tinnitus for those subjects where tinnitus is caused by a loss of non-linear amplification and/or a partial loss of sensitivity.

### I. Neural Substrate

The method of the invention incorporates a minimal assumption on the neural processing that is required during the gain adaptation. That is, an assumption is made that intensity is encoded separately for each frequency band, presumably in neuronal firing rates of a group of neurons, and that the overall loudness of the signal is encoded separately from the intensity of an individual band. Finally, the method includes the assumption that signal power can be accumulated over some time frame and that this estimate can be used to reduce or inhibit the activity in each band. Most of these assumptions are compatible with present knowledge of neuronal function.

The present inventors have also determined that there is no need to specify at which level of neural processing the gain adaptation mechanism may be operating. In fact, the method of the invention can implement several stages of adaptation. The gain control could be operate, for example, as part of the control of outer hair cell response through medial olivocochlear (MOC) efferent feedback. Here, it should be noted that the efferent inhibition of outer hair function as evidenced by distortion products is impaired in most tinnitus subjects. Gain could also be adjusted through inhibition and/or excitation of primary afferent nerve fibers through lateral olivocochlear (LOC) efferents. Finally, adaptation has been demonstrated for inferior colliculus neurons. However, there is some hesitation to include in this list the accommodation observed in auditory nerve fibers in response to a constant stimulus as the effect has been shown to be a subtractive inhibition rather than a divisive normalization. With a time constant of 20 ms or less, it is also faster than the time constant of the Zwicker tone of tinnitus.

Specific evidence for a divisive normalization is sparse. While it has been documented for the visual cortex, the evidence for the auditory stream so far is only indirect. Inhibition in lateral superior ollivary units may have been explained as a divisive process, and inhibition has been shown to mediate gain control in inferior colliculus neurons. In addition, it is suggested that lateral olivocochlear (LOC) efferents are candidates to mediate a divisive/multiplicative gain adaption due to the unique en passant synapses on the afferent dendrites beneath the inner hair cells.

### J. Prediction

As indicated in FIG. 5, the proposed gain adaptation predicts that the masking behavior of the Zwicker tone should vary across frequencies for a given subject, depending on the strength of non-linear compression at each frequency band. Therefore, it is predicted that there is a link between the Zwicker tone masking behavior and the various correlates that are commonly associated with the cochlear amplifier, such as distortion products or two tone suppression—both of these can be measured psychophysically or audiometrically using otoacoustic emissions.

### K. Diagnosis and Treatment Options for Tinnitus

From the preceding discussion it should be apparent that tinnitus occurs because of elevated gains in some central processing stage. These gains, if controlled by a neural gain adaptation mechanism, may be reduced by delivering signal power to the corresponding frequency band in which the elevated gain has occurred. In particular, central gain adaptation will be restored to its normal function if non-linear compression in the damaged frequency band is restored. Fortunately, residual hearing in a damaged frequency band can be augmented using a hearing aid that incorporates the method of the invention, where the hearing aid is appropriately fitted to compensate the specific deficit of the subject. Alternatively to a hearing aid, any calibrated audio device can be used to deliver normal auditory stimuli that are modified to compensate for the specific deficit, such as much, speech or natural sounds.

To facilitated this correction in non-linear compression it is required to measure compression in addition to perception thresholds in separate frequency bands. Here, however, there is advantageously no need to deliver artificial signals or noise signals in the manner of conventional tinnitus or hyperacusis treatment regimes. It is sufficient to appropriately enhance the natural auditory input to the patient.

Tinnitus is associated with long-term adaptive mechanisms. Consequently, it is possible to eliminate the need to constantly apply the method of the invention by way of the hearing aid. Instead, the method of the invention can be implemented in a device selectively, i.e., restricted to limited times of the day, such as during nightly sleep with natural environmental sounds delivered with a corresponding hearing aid. Alternatively one can modifying the sound output of conventional personal electronic devices, such as cell phones, music players (e.g. an ®Apple iPod® or other digital media players) or non-personal electronic devices such as TV or home stereo. This option is particularly appealing as the required correction mechanism could be easily added at the output of existing devices, potentially requiring changes only to the software, or a separate universal add-on device. In all instances, sound should be delivered with earphones or headphones and devices should be of sufficient sound quality to deliver calibrated sound up to high frequency bands (16 kHz).

The method of the invention advantageously implements both amplification and compression. A corresponding diagnosis process (or fitting) is used to determine the optimal parameters of the method for each frequency band for each subject that suffers from tinnitus and/or hyperacusis. The fitting can be accomplished either with psychometric or physiological procedures. Psychometric procedures can include hearing thresholds, loudness growth, two-tone suppression, distortion products, or any other procedure that can reveal the altered amplification and compression processes of impaired hearing. In the specific case of peripheral damage, it is also possible to resort to otoacoustic emissions, such as the input-output growth functions of distortion products and/or spontaneous acoustic emission, in particular involving contralateral stimulation to determine the health of the efferent pathway that modulates the cochlear amplifier. The alteration of these, and other such measures, have been implicated in tinnitus. They are widely used to characterize various forms of hearing loss and loss of non-linear compression, both of which, when restored, should alleviate tinnitus and hyperacusis.

The method of the invention thus provides a simple, optimal auditory adaptation that can account for tinnitus as a consequence of a mismatch between the design parameters of the adaptation to the actual performance of the sensory apparatus.

Thus, while there have been shown, described and pointed out fundamental novel features of the invention as embodied in a method for alleviating tinnitus and hyperacusis by compensating for hearing loss and loss of non-linear compression, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

What is claimed is:

1. A method for alleviating tinnitus and hyperacusis, comprising the steps of:
determining hearing loss and compressive loss of a subject at separate frequency bands;
modifying natural acoustic signals to compensate for compressive loss and loss of hearing sensitivity of the subject at said frequency bands; and
delivering this the modified signal as auditory stimulus to the subject.

2. The method of claim 1, wherein said determining step comprises measuring hearing sensitivity with high frequency resolution audiograms.

3. The method of claim 1, wherein said determining comprises measuring compressive hearing loss of the subject with high frequency resolution distortion product otoacoustic emissions (DPOAE).

4. The method of claim 1 further comprising:
applying therapeutic auditory stimulation temporarily via a conventional audio device.

5. The method of claim 4, wherein the audio device is portable.

6. The method of claim 4, wherein audio device is a portable music player.

7. The method of claim 1, further comprising the step of:
applying therapeutic stimulation chronically via a hearing-aid device.

8. The method of claim 4, further comprising the step of:
synthesizing sounds to deliver sound energy to compensate for specific hearing loss of the subject.

9. The method of claim 8, further comprising the step of:
synthesizing sounds to deliver sound energy to compensate for specific hearing loss of the subject.

10. The method of claim 1, further comprising the step of:
implementing the method during limited times of the day with at least one of natural environmental sounds delivered with a corresponding hearing aid and by modifying sound output by a conventional personal electronic device.

11. The method of claim 10, wherein the limited times of the day is during nightly sleep.

12. The method of claim 1, wherein said step of determining hearing loss and compressive loss of a subject at separate frequency bands comprises:
diagnosing the subject to determine optimal parameters for each frequency band for the subject.

13. The method of claim 12, wherein the diagnosis is performed via psychometric or physiological testing.

14. The method of claim 13, wherein the psychometric testing include at least one of hearing thresholds, loudness growth, two-tone suppression and distortion products.
